(19) United States
(12) Patent Application Publication
(10) Pub. No.: US 2022/0186203 A1
(43) Pub. Date: Jun. 16, 2022

(54) ENDOGLUCANASE VARIANTS WITH IMPROVED PROPERTIES

(71) Applicant: METGEN OY, Kaarina (FI)

(72) Inventors: Klara Birikh, Kaarina (FI); Veera Kaarina Hamalainen, Kaarina (FI)

(21) Appl. No.: 17/442,724

(22) PCT Filed: Mar. 23, 2020
(86) PCT No.: PCT/EP2020/057919

§ 371 Date: Sep. 24, 2021

(30) Foreign Application Priority Data
Mar. 26, 2019 (EP) 19165243.7

(51) Int. Cl. C12N 9/96; C12N 15/70; C12N 15/75; C12N 15/81; C12N 9/42

(52) U.S. Cl. CPC C12N 9/96; C12N 15/70; C12N 15/75; C12N 2800/10; C12N 9/2437; C12Y 302/01004; C12N 15/815

(57) ABSTRACT

The present invention relates to endoglucanase variants with improved properties and uses thereof as eco-friendly biocatalysts in various industrial processes. More in particular the invention provides a polypeptide with endoglucanase activity (EC 3.2.1.4) comprising an amino acid sequence that is at least 90% identical to the amino acid sequence according to SEQ ID NO: 1 or SEQ ID NO: 2, wherein the polypeptide comprises an amino acid selected from the group consisting of Glycine, Threonine, Serine, Alanine, Leucine, Isoleucine and Valine at a position corresponding to position 579 in SEQ ID NO: 1 or SEQ ID NO: 2 or an amino acid selected from the group consisting of Serine, Alanine and Valine at a position corresponding to position 656 in SEQ ID NO: 1 or SEQ ID NO: 2.

### CROSS REFERENCE TO RELATED APPLICATIONS

This application is a national phase entry under 35 U.S.C. § 371 of International Patent Application PCT/EP2020/057919, filed Mar. 23, 2020, designating the United States of America and published in English as International Patent Publication WO 2020/193452 on Oct. 1, 2020, which claims the benefit under Article 8 of the Patent Cooperation Treaty to European Patent Application Serial No. 19165243.7, filed Mar. 26, 2019, the entireties of which are hereby incorporated by reference.

### FIELD OF THE INVENTION

The present invention relates to endoglucanase variants with improved properties and uses thereof as eco-friendly biocatalysts in various industrial processes.

### BACKGROUND OF THE INVENTION

Endoglucanases (EC 3.2.1.4) are also known as endo-1,4-beta-glucanase, Beta-1,4-endoglucan hydrolase, beta-1,4-glucanase, carboxymethyl cellulase, celludextrinase, endo-1,4-beta-D-glucanase or endo-1,4-beta-D-glucanohydrolase. They cleave internal glycoside bonds in a glucose polymer, as opposed to exoglucanases, which clip off a cellobiose from one end of a glucose polymeric chain. Exoglucanases are also known as exo-cellobiohydrolase; beta-1,4-glucan cellobiohydrolase; beta-1,4-glucan cellobiosylhydrolase; 1,4-beta-glucan cellobiosidase; exoglucanase or avicelase.

Cellulose, a polysaccharide of β-1,4-linked d-glucose units, is the most abundant biopolymer on earth. It is the main constituent of plant cell walls, where it forms a tight complex together with hemicelluloses and is embedded in the lignin matrix. Cellulose is a recalcitrant material organized into microfibrils and composed of highly ordered glucan chains interlinked by hydrogen bonds. Depending on the source of cellulose, these fibrils have a more or less crystalline character, and for deconstruction of these complex structures, microorganisms have developed specialized enzymatic systems. All cellulolytic organisms produce multiple enzymes for cellulose degradation, but three main catalytic activities are necessary for complete hydrolysis: exoglucanases (or cellobiohydrolases [CBHs]) attack cellulose chains from the chain ends, and endoglucanases cleave the cellulose chain randomly, while β-glucosidases hydrolyze cellobiose, the reaction product of cellobiohydrolases. For efficient degradation, all three enzymatic activities have to be present (Poidevin et al., Applied Environmental Microbiol 79; 4220-4229 (2013) DOI: 10.1128/AEM.00327-13).

For industrial applications it is useful to perform degradation of cellulose at elevated temperatures, such as 40 degrees Celsius or above. Typical industrial processes, such as degradation of wood chips in the pulp and paper industry, work at 40 degrees Celsius.

So far only a few thermophilic microorganisms are reported to be able to grow on cellulose as the sole substrate. A hypothetical glycoside hydrolase has been described to be encoded by the genome of Spirochaeta thermophila (WP_013314422.1). This enzyme has not been expressed so far, nor has its activity been experimentally characterised.

Recombinant protein expression in easily cultivatable hosts can allow higher productivity in shorter time and reduces the costs of production. The versatility and scaling-up possibilities of recombinant protein production opened up new commercial opportunities for their industrial uses. Moreover, protein production from pathogenic or toxin-producing species can take advantage of safer or even GRAS (generally recognized as safe) microbial hosts. In addition, protein engineering can be employed to improve the stability, activity and/or specificity of an enzyme, thus tailor made enzymes can be produced to suit the requirement of the users or of the process.

In general, enzyme productivity can be further increased by the use of multiple gene copies, strong promoters and efficient signal sequences, properly designed to address proteins to the extracellular medium, thus simplifying downstream processing.

There is a need in the art for highly active endoglucanases that function at elevated temperatures, such as 40 degrees Celsius and preferably also are stable at elevated temperatures, even up to 70 or 80 degrees Celsius and higher.

### SUMMARY OF THE INVENTION

The present invention addresses this need in that it provides a polypeptide with endoglucanase activity (EC 3.2.1.4)) comprising an amino acid sequence that is at least 90% identical to the amino acid sequence according to SEQ ID NO: 1 or SEQ ID NO: 2, wherein the polypeptide comprises an amino acid selected from the group consisting of Glycine, Threonine, Serine, Alanine, Leucine, Isoleucine and Valine at a position corresponding to position 579 in SEQ ID NO: 1 or SEQ ID NO: 2 or an amino acid selected from the group consisting of Serine, Alanine and Valine at a position corresponding to position 656 in SEQ ID NO: 1 or SEQ ID NO: 2.

The invention also provides a DNA encoding such a polypeptide, a vector and a recombinant expression system that produces an enzyme according to the invention.

Also provided herein is the use of a polypeptide according to the invention in an application selected from the group consisting of paper production, cardboard production, nanocellulose production, biorefinery applications, cellulose hydrolysis, wood fiber modification, wood pulp delignification for improving wood fiber properties, improving paper strength, degrading or decreasing the structural integrity of lignocellulosic material, production of a sugar from a lignocellulosic material and depolymerizing cellulose, such as cellulose from a biomass.

Also provided herein is a method for improving the specific activity of a polypeptide with endoglucanase activity in a heterologous expression system comprising a step of altering an amino acid at a position corresponding to position 579 into a Glycine, Threonine, Serine, Alanine, Leucine, Isoleucine or Valine and/or altering an amino acid at a position corresponding to position 656 into a Serine, Alanine or Valine, wherein the polypeptide with endoglucanase activity is a polypeptide with an amino acid sequence according to SEQ ID NO: 1 or SEQ ID NO: 2 or a polypeptide with an amino acid sequence that is at least 90% identical to the sequence of SEQ ID NO: 1 or SEQ ID NO: 2.

### DETAILED DESCRIPTION OF THE INVENTION

We cloned and expressed a DNA sequence according to SEQ ID NO: 9 and obtained a polypeptide according to SEQ ID NO: 1 (Table 1) which appeared to have endoglucanase activity when tested in an assay as described in example 4.

SEQ ID NO: 1 is similar to a sequence obtained from a public database obtainable from the National Center for Biotechnology Information (NCBI) Reference number: WP_013314422.1.

The endoglucanase according to SEQ ID NO: 1 appeared to be active at 40 degrees Celsius and very stable in that it retained more than 95%, such as 96%, 97%, 98% or even 99% or 100% of its activity after incubation with substrate for at least 72 hours at 40 degrees Celsius. The endoglucanase according to SEQ ID NO: 1 was also very thermoresistant in that it retained more than 95% such as 96%, 97%, 98% or even 99% or 100% of its activity (measured at 40 degrees Celsius) after heating to 70 degrees Celsius for 20 minutes followed by cooling down to 40 degrees Celsius.

We also cloned and expressed an analogue of the polypeptide according to SEQ ID NO: 1 which was 94.3% identical to SEQ ID NO: 1. This sequence is shown herein as SEQ ID NO: 2. The DNA encoding SEQ ID NO: 2 is shown herein as SEQ ID NO: 10 (Table 1).

SEQ ID NO: 2 is similar to a sequence obtained from a public database obtainable from the National Center for Biotechnology Information (NCBI) Reference number: WP_014625293.1.

The endoglucanase according to SEQ ID NO: 2 also appeared to be active at 40 degrees Celsius and very stable in that it retained more than 95%, such as 96%, 97%, 98% or even 99% or 100% of its activity after incubation with substrate for at least 72 hours at 40 degrees Celsius. The endoglucanase according to SEQ ID NO: 2 was also very thermoresistant in that it retained more than 95% such as 96%, 97%, 98% or even 99% or 100% of its activity (measured at 40 degrees Celsius) after heating to 70 degrees Celsius for 20 minutes followed by cooling down to 40 degrees Celsius.

We also provide herein mutants or variants of endoglucanases according to SEQ ID NO: 1 and SEQ ID NO: 2 with an improved or increased specific activity.

As used herein, the term “specific activity” refers to the activity per weight of an enzyme. Increased or improved specific activity of a mutated or variant endoglucanase refers to an endoglucanase activity higher than that of a corresponding non-mutated endoglucanase enzyme under the same conditions for the same amount of enzyme, expressed as weight of endoglucanase protein. That improvement can be as high as at least 50%, such as at least 100%, 150%, 200%, 400%, or more. We also found that the mutated endoglucanases retain the thermostability of the wild type protein.

The phrase “amino acid at a position corresponding to position xxx”, as used herein, wherein xxx is a number, is to be interpreted as follows. In order to determine whether an amino acid at a certain position in a first amino acid sequence corresponds to a certain amino acid in a second amino acid sequence, the first and second amino acid sequences first have to be aligned using standard software available in the art, such as the “Align” tool at NCBI recourse http://blast.ncbi.nlm.nih.gov/Blast.cgi?PAGE_TYPE=BlastSearch&BLAST_SPEC=blast2s eq&LINK_LOC=align2seq. The corresponding amino acid positions then follow from that alignment.

The terms “variant”and “mutant” are used herein interchangeably and indicate a deviation from the wild type sequence.

Endoglucanases according to SEQ ID NO: 1 and SEQ ID NO: 2 are referred herein as the wild type sequences, whereas the SEQ ID NO:s 3-8 are herein referred to as variants or mutants. More in particular, we provide herein variants of an endoglucanase according to SEQ ID NO:1 or SEQ ID NO: 2 with an improved or increased specific activity due to at least one point mutation. These mutant endoglucanases share the technical feature that they have an increased specific activity at 40 degrees Celsius as compared to the specific activity of their wild type polypeptide according to SEQ ID NO: 1 or SEQ ID NO: 2.

TABLE 1
Protein and DNA sequences of wild type endoglucanases as described herein

| SEQ | |
|---|---|
| ID | |
| NO: | Sequence |
| 1 | MVPEGGGKAPPEEAGETAAEELLENGDFSAGTTPWTLFTQDPGKAVMTTEDGMLVFEISSIGEDTWHVQPGYPGLHLEEGHTYRLEFDMRASEPRTVQ |
| | VRIQKDGPPWTGYLEENFQVGTALQHYTFEFSMQETDKAARLVFNLGTAAEGTAPAGAHRIYLDNISLKDLTGGPPEEKTGGLRPAVHLNQVGYLPTA |
| | PKVFVTMVDSSSFKVLEADSGKEVFSGTLSSPIPDRDSGDTVRLGDFTALTTPGTYVVEVGETRSVPFEIREDIYEELHTALFRFFYLQRCGTELAPS |
| | LAGAWAHPACHTTPALVYGTTITKEVRGGWHDAGDYGRYVVPAAKAVADLLLAHLFYPNATSSDALEIPESGNGVPDVLDEVKDELLWLLSMQDPESG |
| | GVYHKVTTRNFAGFDWPHITGGELVLSPISSAATADFAAVMAMASRVYAIVEPRLARRALEAARRAWSWLEAHPSAPGFRNPPGIQTGEYGDDHDADE |
| | RYWAACELYAATGEEDFHTALKALSRGDIRAGLGWADVADYGTITYLFFTPHKDEALAAELAAHLTAKAEAILATMETSGYRISLTEYPWGSNMTVAN |
| | NGMYLLIASRLTGDPRYERAAAEHLDYLLGRNPLSRSYITGFGEKAAEHPHHRVSSAAGTTIPGMVVGGPNSGLQDPVAQGALRGEPPAKCYIDDVGS |
| | YSTNEVTIYWNSPVYFLVSGLVE |
| 2 | MVPERRGEAPPEEAGETAAEELLENGDFSAGTTPWTLFTQDPGKAVMTTEDGMLVFEISSIGEDTWHVQPGYPGLHLIEGHTYRLEFDMRASEPRTVQ |
| | VRIQKDGPPWTGYLEENFQVGNEFQHYTFEFTMQETDKAARLVFNLGTAAEGTAPAGPHRVSLDNISLKDLTGGPPEEETGGLRPAVHLNQLGYLPTA |
| | PKVFVTTVDSSSFKVLEADSGKEVFSGTLSSPIPDRDSGDTVRVGDFTALTTPGTYVVEAGETRSVPFEIREDIYEELHTALFRFFYLQRCGTELASS |
| | LAGAWAHPACHTAPALVYGTTITKEVRGGWHDAGDYGRYVVPAAKAVADIILAHLFYPDATSSDALEIPESGNGVPDVLDEVRDELLWLLSMQDPEGG |
| | GVYHKVTTRNFAGFDWPQNTGGELVLSPISPTATADFAAVMAMASRVYASIDPGLARRALEAARRAWSWLEAHPSAPGFRNPPGIQTGEYGDDHDADE |
| | RYWAACELYAATGEEDFHAALEGLSRGDLRAGLGWADVADYGTITYLFFTPHKDEALAAELAAHLTDKAEAILATMETSGYRTSLTEYPWGSNMTVAN |
| | NGMYLLITSRLTGDPRYERAAAEHLDYLLGRNPLSRSYITGFGEKVAEHPHHRVSSAAGMTIPGMVVGGPNSGLQDPVAQGALRGEPPAKCYIDDVGS |
| | YSTNEVTIYWNSPVYFLVSGLVE |
| 9 | ATGGTTCCGGAAGGTGGTGGTAAAGCACCGCCTGAAGAAGCAGGCGAAACCGCAGCCGAAGAACTGCTGGAAAATGGTGATTTTAGCGCAGGCACCAC |
| | CCCGTGGACCCTGTTTACCCAAGATCCGGGTAAAGCAGTTATGACCACCGAAGATGGTATGCTGGTTTTTGAAATTAGCAGCATTGGTGAAGATACCT |
| | GGCATGTTCAGCCTGGTTATCCGGGTCTGCATCTGGAAGAAGGTCATACCTATCGTCTGGAATTTGATATGCGTGCAAGCGAACCGCGTACCGTTCAG |
| | GTTCGTATTCAGAAAGATGGTCCGCCTTGGACCGGTTACCTGGAAGAAAACTTTCAGGTTGGCACCGCACTGCAGCATTATACCTTTGAATTTAGCAT |
| | GCAAGAAACCGATAAAGCAGCACGTCTGGTTTTTAATCTGGGCACCGCAGCAGAAGGTACAGCACCGGCAGGCGCACATCGTATTTATCTGGATAACA |
| | TTAGCCTGAAAGATCTGACCGGTGGTCCTCCGGAAGAAAAAACCGGTGGCCTGCGTCCGGCAGTTCATCTGAATCAGGTTGGTTATCTGCCGACCGCA |
| | CCGAAAGTTTTTGTTACAATGGTTGATAGCAGCAGCTTCAAAGTTCTGGAAGCAGATAGCGGTAAAGAAGTTTTTAGCGGCACCCTGAGCAGCCCGAT |
| | TCCGGATCGTGATAGCGGTGATACCGTTCGTCTGGGCGATTTTACCGCACTGACCACACCGGGTACATATGTTGTTGAAGTTGGTGAAACCCGTAGCG |
| | TTCCGTTTGAAATTCGTGAAGATATCTATGAGGAACTGCATACAGCACTGTTCCGTTTTTTCTATCTGCAGCGTTGTGGCACCGAACTGGCACCGAGC |
| | CTGGCTGGTGCATGGGCACATCCGGCATGTCATACAACACCGGCACTGGTTTATGGCACCACCATTACCAAAGAAGTTCGCGGAGGTTGGCATGATGC |
| | CGGTGATTATGGTCGTTATGTTGTTCCGGCAGCAAAAGCAGTTGCCGATCTGCTGCTGGCACACCTGTTTTATCCGAATGCAACCAGCAGTGATGCAC |
| | TGGAAATTCCGGAAAGCGGTAATGGCGTTCCGGATGTTCTGGATGAAGTTAAAGATGAGCTGCTGTGGCTGCTGAGCATGCAGGACCCTGAAAGCGGT |
| | GGTGTTTATCATAAAGTTACCACCCGTAATTTTGCCGGTTTTGATTGGCCTCATATTACCGGTGGTGAACTGGTTCTGAGCCCGATTAGCAGCGCAGC |
| | AACCGCAGATTTTGCAGCCGTTATGGCAATGGCAAGCCGTGTTTATGCAATTGTTGAACCGCGTCTGGCACGTCGTGCCCTGGAAGCTGCTCGTCGTG |
| | CATGGTCATGGCTGGAAGCACATCCGAGCGCACCGGGTTTTCGTAATCCGCCTGGTATTCAGACAGGTGAATATGGTGATGATCATGATGCGGATGAA |
| | CGTTATTGGGCAGCATGTGAACTGTATGCCGCAACCGGTGAAGAAGATTTTCATACAGCGCTGAAAGCACTGAGCCGTGGTGATATTCGTGCAGGTCT |
| | GGGTTGGGCAGATGTTGCAGATTATGGTACAATTACCTACCTGTTTTTCACACCGCATAAAGATGAAGCACTGGCAGCAGAACTGGCAGCCCATCTGA |
| | CCGCAAAAGCCGAAGCAATTCTGGCAACAATGGAAACCAGCGGTTATCGTATTAGTCTGACCGAATATCCGTGGGGTAGCAATATGACCGTTGCAAAT |
| | AATGGTATGTATCTGCTGATTGCAAGCCGTCTGACAGGTGATCCGCGTTATGAACGTGCAGCAGCCGAACATCTGGATTATCTGCTGGGTCGCAATCC |
| | GCTGAGCCGTAGCTATATTACAGGTTTTGGTGAAAAAGCTGCGGAACATCCTCATCATCGTGTTTCAAGCGCAGCCGGTACAACCATTCCGGGTATGG |
| | TGGTTGGTGGTCCGAATAGCGGTCTGCAAGATCCTGTTGCACAGGGTGCACTGCGTGGTGAACCGCCTGCAAAATGTTATATTGATGATGTTGGTAGC |
| | TACAGCACCAACGAAGTTACCATTTATTGGAATAGTCCGGTGTATTTTCTGGTGAGCGGTCTGGTTGAA |
| 10 | ATGGTTCCGGAACGTCGTGGTGAAGCACCGCCTGAAGAAGCAGGCGAAACCGCAGCCGAAGAACTGCTGGAAAATGGTGATTTTAGCGCAGGTACAAC |
| | CCCGTGGACACTGTTTACCCAAGATCCGGGTAAAGCAGTTATGACCACCGAAGATGGTATGCTGGTTTTTGAAATTAGCAGCATTGGTGAAGATACCT |
| | GGCATGTTCAGCCTGGTTATCCGGGTCTGCATCTGATTGAAGGTCATACCTATCGTCTGGAATTTGATATGCGTGCAAGCGAACCGCGTACCGTTCAG |
| | GTTCGTATTCAGAAAGATGGTCCGCCTTGGACCGGTTATCTGGAAGAAAACTTTCAGGTGGGTAACGAATTTCAGCACTATACCTTTGAATTTACCAT |
| | GCAAGAAACCGATAAAGCAGCACGTCTGGTTTTTAATCTGGGCACCGCAGCAGAAGGCACCGCACCGGCAGGTCCGCATCGTGTTAGCCTGGATAACA |
| | TTAGCCTGAAAGATCTGACCGGTGGTCCTCCGGAAGAAGAAACAGGCGGTCTGCGTCCGGCAGTTCATCTGAATCAGCTGGGTTATCTGCCGACCGCA |
| | CCGAAAGTGTTTGTTACCACCGTTGATAGCAGCAGCTTTAAAGTTCTGGAAGCAGATAGCGGTAAAGAAGTTTTTAGCGGCACCCTGAGCAGCCCGAT |
| | TCCGGATCGTGATAGCGGTGATACCGTTCGTGTTGGCGATTTTACCGCACTGACCACACCGGGTACATATGTTGTTGAAGCCGGTGAAACCCGTAGCG |
| | TTCCGTTTGAAATTCGCGAAGATATTTATGAGGAACTGCATACAGCACTGTTCCGCTTTTTCTATCTGCAGCGTTGTGGCACCGAACTGGCAAGCAGC |
| | CTGGCAGGCGCATGGGCACATCCGGCATGTCATACGGCACCGGCACTGGTTTATGGCACCACCATTACCAAAGAAGTTCGCGGAGGTTGGCATGATGC |
| | CGGTGATTATGGTCGTTATGTTGTTCCGGCAGCAAAAGCAGTTGCAGATATTATTCTGGCACACCTGTTTTATCCGGATGCAACCAGCAGTGATGCAC |
| | TGGAAATTCCGGAAAGCGGTAATGGCGTTCCGGATGTTCTGGATGAAGTTCGTGATGAGCTGCTGTGGCTGCTGAGCATGCAGGACCCTGAAGGTGGT |
| | GGTGTTTATCATAAAGTTACCACACGTAACTTTGCCGGTTTTGATTGGCCTCAGAATACCGGTGGTGAACTGGTTCTGAGCCCGATTAGCCCGACCGC |
| | AACCGCAGATTTTGCAGCCGTTATGGCAATGGCAAGCCGTGTTTATGCAAGCATTGATCCTGGTCTGGCACGTCGTGCCCTGGAAGCTGCTCGTCGTG |
| | CATGGTCATGGCTGGAAGCACATCCGAGCGCACCGGGTTTTCGTAATCCGCCTGGTATTCAGACAGGTGAATATGGTGATGATCATGATGCGGATGAA |
| | CGTTATTGGGCAGCATGTGAACTGTATGCCGCAACCGGTGAAGAAGATTTTCACGCAGCACTGGAAGGTCTGAGCCGTGGTGATCTGCGTGCAGGTTT |
| | AGGTTGGGCAGATGTTGCAGATTATGGTACAATTACCTACCTGTTTTTCACACCGCATAAAGATGAAGCACTGGCAGCAGAACTGGCAGCCCATCTGA |
| | CAGATAAAGCCGAAGCAATTCTGGCAACAATGGAAACCAGCGGTTATCGTACCAGCCTGACCGAATATCCGTGGGGTAGCAATATGACCGTTGCAAAT |
| | AATGGTATGTATCTGCTGATTACCAGCCGTCTGACAGGTGATCCGCGTTATGAACGTGCAGCAGCCGAACATCTGGATTATCTGCTGGGTCGCAATCC |
| | GCTGAGCCGTAGCTATATTACCGGTTTTGGTGAAAAAGTTGCGGAACATCCTCATCATCGTGTGAGCAGCGCAGCCGGTATGACCATTCCTGGTATGG |
| | TTGTTGGTGGTCCGAATAGCGGTCTGCAAGATCCTGTTGCACAGGGTGCACTGCGTGGTGAACCGCCTGCAAAATGTTATATTGATGATGTTGGTAGC |
| | TACAGCACCAACGAAGTTACCATTTATTGGAATAGTCCGGTGTATTTTCTGGTGAGCGGTCTGGTTGAA |

More in particular, the invention relates to a polypeptide with endoglucanase activity comprising the amino acid sequence according to SEQ ID NO: 1 or SEQ ID NO: 2, or an amino acid sequence that is at least 90% identical with SEQ ID NO: 1 or SEQ ID NO: 2, wherein the polypeptide comprises an amino acid selected from the group consisting of Glycine, Threonine, Serine, Alanine, Leucine, Isoleucine and Valine at a position corresponding to position 579 in SEQ ID NO: 1 or SEQ ID NO: 2 and/or an amino acid selected from the group consisting of Serine, Alanine and Valine at a position corresponding to position 656 in SEQ ID NO: 1 or SEQ ID NO: 2.

Single amino acid mutations at position 579 are represented by SEQ ID NO:s 5-8, whereas double mutations are represented by SEQ ID NO:s 3 and 4.

The individual variants are herein referred to as follows. When referring to substitutions in a polypeptide according to SEQ ID NO: 1: the annotation G656S refers to a substitution of Glycine at position 656 in SEQ ID NO: 1 with an Serine.

In other words, the invention relates to a polypeptide with endoglucanase activity (EC 3.2.1.4)) comprising an amino acid sequence that is at least 90% identical to the amino acid sequence according to SEQ ID NO: 1 or SEQ ID NO: 2, wherein the polypeptide comprises an amino acid selected from the group consisting of Glycine, Threonine, Serine, Alanine, Leucine, Isoleucine and Valine at a position corresponding to position 579 in SEQ ID NO: 1 or SEQ ID NO: 2 or an amino acid selected from the group consisting of Serine, Alanine and Valine at a position corresponding to position 656 in SEQ ID NO: 1 or SEQ ID NO: 2.

All single and double mutants showed an increased relative activity when compared to the wild type activity as is shown in tables 2 and 3. This is due to an increase in specific activity rather than an increase in volumetric activity since all mutants expressed the same amount of protein when analysed by SDS PAGE (Example 4).

TABLE 2
Relative specific activity (in % relative to
wild type SEQ ID NO: 1) of mutant
endoglucanases of SEQ ID NO: 1

| | WT G656 | G656S | G656V | G656A |
|---|---|---|---|---|
| WT P579 | 100 | 200 | 200 | 180 |
| P579G | 210 | 390 | 380 | 360 |
| P579T | 170 | 350 | 355 | 340 |
| P579S | 152 | 345 | 340 | 320 |
| P579A | 160 | 355 | 360 | 335 |
| P579L | 170 | 360 | 350 | 340 |
| P579I | 165 | 350 | 340 | 320 |
| P579V | 161 | 355 | 345 | 325 |

TABLE 3
Relative specific activity (in % relative to
wild type SEQ ID NO: 2) of mutant
endoglucanases of SEQ ID NO: 2.

| | WT G656 | G656S | G656V | G656A |
|---|---|---|---|---|
| WT P579 | 100 | 210 | 210 | 170 |
| P579G | 215 | 395 | 380 | 350 |
| P579T | 165 | 355 | 375 | 330 |
| P579S | 158 | 340 | 360 | 310 |
| P579A | 155 | 350 | 330 | 325 |
| P579L | 172 | 366 | 360 | 330 |
| P579I | 169 | 340 | 345 | 310 |
| P579V | 156 | 345 | 340 | 315 |

In addition, the invention provides a composition comprising the above polypeptides, as well as nucleic acids, vectors and compositions comprising such nucleic acids encoding the endoglucanase enzymes according to the invention.

The invention also provides recombinant heterologous expression systems such as host cells comprising a nucleic acid or a vector according to the invention.

The invention also provides a method for producing a polypeptide according to the invention, comprising the steps of:
a. culturing a recombinant host cell under conditions suitable for the production of the polypeptide, and
b. recovering the polypeptide obtained, and
c. optionally purifying the polypeptide.

The invention also provides the use of a polypeptide as described herein in an application selected from the group consisting of paper production, cardboard production, nanocellulose production, biorefinery applications, cellulose hydrolysis, wood fiber modification, wood pulp delignification for improving wood fiber properties, improving paper strength, degrading or decreasing the structural integrity of lignocellulosic material, production of a sugar from a lignocellulosic material and depolymerizing cellulose, such as cellulose from a biomass.

The invention also provides a method for improving or increasing the specific activity of a polypeptide with endoglucanase activity in a heterologous expression system comprising the step of altering the amino acid corresponding to the amino acid at position 579 in SEQ ID NO: 1 or SEQ ID NO: 2 into an amino acid selected from the group consisting of a Glycine, Threonine, Serine, Alanine, Leucine, Isoleucine or Valine, and/or altering the amino acid corresponding to the amino acid at position 656 in SEQ ID NO: 1 or SEQ ID NO: 2 into an amino acid selected from the group consisting of Serine, Alanine and Valine, wherein the polypeptide with endoglucanase activity is a polypeptide with an amino acid sequence according to SEQ ID NO: 1 or SEQ ID NO: 2 or a polypeptide with an amino acid sequence that is at least 90% identical to the sequence of SEQ ID NO: 1 or SEQ ID NO: 2.

Preferred embodiments of these aspects will be described in more detail below. For the purpose of clarity and a concise description, features are described herein as part of the same or separate embodiments, however, it will be appreciated that the scope of the invention may include embodiments having combinations of all or some of the features described.

Polypeptides according to SEQ ID NO: 1 and SEQ ID NO: 2 were expressed in good yield in recombinant hosts such as E. coli, Bacillus subtilis and Pichia pastoris. All showed high thermostability and endoglucanase activity. The enzymes thereby showed high potential to be used in wood fiber modification for improved paper properties and other applications.

The term “endoglucanase activity” is used herein to mean the property of a polypeptide to act as a endoglucanase enzyme, which may be expressed as the maximal initial rate of cleavage of glycosidic 1-4 bonds (example 4).

As used herein, the term “specific activity” refers to the activity per weight of an enzyme. Increased or improved specific activity of a mutated or variant endoglucanase refers to an edoglucanase activity higher than that of a corresponding non-mutated endoglucanase enzyme under the same conditions for the same amount, expressed as weight of endoglucanase protein.

As used herein, the term “Volumetric activity” refers to the activity per volume unit of the production culture. Increased or improved volumetric activity of a mutated or variant endoglucanase refers to an endoglucanase activity higher than that of a corresponding non-mutated endoglucanase enzyme obtained from the same production culture volume.

As used herein, the term “relative activity” (expressed in %) refers to the fraction of enzyme activity as compared to the control enzyme, whereas the activity of the control enzyme in the same volume or weight is taken as 100%.

The term “amino acid substitution” or “amino acid substitutions” is used herein in the same way as it is commonly used, i.e. the term refers to a replacement of one or more amino acids in a protein, at a certain position, with another amino acid. Such amino acid substitutions may also be referred to as “variants” or “mutations”.

The term “amino acid variant”, “variant” or “sequence variant” or “mutant” or equivalent has a meaning well recognized in the art and is accordingly used herein to indicate an amino acid sequence that differs from another amino acid sequence by at least one amino acid.

When the variants according to SEQ ID NO: 3-8 were expressed in Pichia pastoris, the eukaryotic expression also showed an increase in specific activity for all variants. The specific activity of each single and double mutant increased with at least 50%.

When the variants according to SEQ ID NO: 3-8 were also expressed in Bacillus subtilis, the expressed endoglucanases also showed an increase in specific activity for all variants. The specific activity of each single and double mutant increased with at least 50%.

The teaching as provided herein should not be so narrowly construed as that it relates only to the exemplified sequences of SEQ ID NO: 1 or SEQ ID NO: 2 and their variant polypeptides. It is well known in the art that protein sequences may be altered or optimized, for instance by site-directed mutagenesis, in order to arrive at related proteins with identical or even improved properties.

We performed a homology search for proteins homologous to SEQ ID NO: 1 using SEQ ID NO: 1 as the query sequence in the “Standard protein BLAST” software, available at http://blast.ncbi.nlm.nih.gov/Blast.cgi?PROGRAM=blastp&PAGE_TYPE=BlastSearch&LIN K_LOC=blasthome. More information on the software and database versions is available at the National Center for Biotechnology Information at National Library of Medicine at National Institute of Health internet site www.ncbi.nlm.nih.gov. Therein, a number of molecular biology tools including BLAST (Basic Logical Alignment Search Tool) is to be found. The search as reported herein was performed online on 5 Mar. 2016.

The search revealed that the closest homologue of SEQ ID NO: 1 has 45.17% identity to SEQ ID NO: 1. This sequence was from a glycosyl hydrolase family 9 [unclassified Ruminococcaceae (miscellaneous)] Sequence ID: WP_109643445.1

So it may be concluded that there are no endoglucanases known to exist in nature or anywhere else with an amino acid sequence that is more than 90% identical to the endoglucanase according to SEQ ID NO: 1. Nevertheless, such proteins may now be artificially constructed and expressed in a recombinant expression system. It is well within reach of the skilled person to construct such closely related proteins with a sequence identity of 90% or more, such as 93%, 94%, 95%, 96%, 97%, 98% or 99% or more and test them for endoglucanase activity.

Introduction of a specific variation in a recombinant gene is among the routine skills of a molecular biologist. Comprehensive guidance may be obtained from Methods in Molecular Biology Vol 182, “In vitro mutagenesis protocols”, Eds Jeff Braman, Humana Press 2002. There are commercially available kits for performing site-directed mutagenesis (for example, QuikChange II XL Site-Directed Mutagenesis kit Agilent Technologies cat No 200521).

Hence, the invention relates to a variant polypeptides having endoglucanase activity, homologues thereof and methods for their use and production as described herein, wherein the variant polypeptide comprises or consists of an amino acid sequence at least 90% identical to SEQ ID NO: 1 or SEQ ID NO: 2. The term “at least 90%”, is to be interpreted as 90%, 91, 92, 93, 94, 95, 96, 97, 98, 99% or more.

The endoglucanases as presented herein share the technical feature that they have an improved specific activity when expressed in a recombinant host cell such as E. coli, B. subtilis or Pichia as compared to the specific activity of a polypeptide according to SEQ ID NO: 1 or SEQ ID NO: 2.

The endoglucanase variants according to the present invention may be used in a wide range of different industrial processes and applications The invention also provides the use of a polypeptide as described herein in an application selected from the group consisting of wood fiber modification, biomass hydrolysis, cellulose microfibrilation and production of a sugar from a lignocellulosic material.

Amino acid variations as described herein may be introduced into any of the amino acid sequences disclosed herein, or other homologous sequences, by standard methods known in the art, such as site-directed mutagenesis. In this way, the specific activity of the endoglucanase from a heterologous expression system may be improved.

Kits for performing site-directed mutagenesis are commercially available in the art (e.g. QuikChange® II XL Site-Directed Mutagenesis kit by Agilent Technologies). Further suitable methods for introducing the above mutations into a recombinant gene are disclosed e.g. in Methods in Molecular Biology, 2002 [8].

The term “heterologous expression system” or “recombinant expression system” or equivalent means a system for expressing a DNA sequence from one host organism in a recipient organism from a different species or genus than the host organism. The most prevalent recipients, known as heterologous expression systems, are chosen usually because they are easy to transfer DNA into or because they allow for a simpler assessment of the protein's function. Heterologous expression systems are also preferably used because they allow the upscaling of the production of a protein encoded by the DNA sequence in an industrial process. Preferred recipient organisms for use as heterologous expression systems include bacterial, fungal and yeast organisms, such as for example Escherichia coli, Bacillus, Corynebacterium, Pseudomonas, Pichia pastoris, Saccharomyces cerevisiae, Yarrowia lipolytica, filamentus fungi and many more systems well known in the art.

As used herein, the degree of identity between two or more amino acid sequences is equivalent to a function of the number of identical positions shared by the sequences (i.e., % identity=number of identical positions divided by the total number of positions×100), excluding gaps, which need to be introduced for optimal alignment of the two sequences, and overhangs. The comparison of sequences and determination of percent identity between two or more sequences can be accomplished using standard methods known in the art. For example, a freeware conventionally used for this purpose is “Align” tool at NCBI recourse http://blast.ncbi.nlm.nih.gov/Blast.cgi?PAGE_TYPE=BlastSearch&BLAST_SPEC=blast2seq&LINK_LOC=align2seq

In a preferred embodiment the alignment of two sequences is to be performed over the full length of the polypeptides.

The variant endoglucanase polypeptides or proteins as disclosed herein may be fused to additional sequences, such as for instance by attaching or inserting sequences encoding affinity tags, thereby facilitating protein purification (S-tag, maltose binding domain, chitin binding domain). Domains or sequences assisting folding (such as thioredoxin domain, SUMO protein), sequences affecting protein localization (periplasmic localization signals etc), proteins bearing additional function, such as green fluorescent protein (GFP), or sequences representing another enzymatic activity may also be attached. Other suitable fusion partners for the present endoglucanases are known to those skilled in the art.

The present invention also relates to polynucleotides encoding any of the endoglucanase variants disclosed herein. Means and methods for cloning and isolating such polynucleotides are well known in the art.

Furthermore, the present invention relates to a vector comprising a polynucleotide according to the invention, optionally operably linked to one or more control sequences. Suitable control sequences are readily available in the art and include, but are not limited to, promoter, leader, polyadenylation, and signal sequences.

endoglucanase variants according to various embodiments of the present invention may be obtained by standard recombinant methods known in the art. Briefly, such a method may comprise the steps of i) culturing a desired recombinant host cell under conditions suitable for the production of a present endoglucanase polypeptide variant, and ii) recovering the polypeptide variant obtained. The polypeptide may then optionally be further purified.

A large number of vector-host systems known in the art may be used for recombinant production of endoglucanase variants. Possible vectors include, but are not limited to, plasmids or modified viruses which are maintained in the host cell as autonomous DNA molecule or integrated in genomic DNA. The vector system must be compatible with the host cell used as is well known in the art. Non-limiting examples of suitable host cells include bacteria (e.g. E. coli, bacilli), yeast (e.g. Pichia pastoris, Saccharomyces cerevisae), fungi (e.g. filamentous fungi) insect cells (e.g. Sf9).

In yet other terms, the invention relates to a method for improving the specific activity of a polypeptide with endoglucanase activity in a heterologous expression system comprising the step of altering at least one amino acid at a position corresponding to position 579 and/or 656 to one of the amino acids as disclosed herein.

### EXAMPLES

### Example 1

### Heterologous Expression of Variant and Non-Mutated Endoglucanases

Recombinant endoglucanases were expressed in E. coli, Bacillus subtilis and Pichia pastoris. Genes according to SEQ ID NO: 9 and SEQ ID NO: 10 were synthesized chemically and mutations were introduced as described in example 2.

The coding sequences of recombinant endoglucanases were cloned into the pHT43 vector for expression in Bacillus subtilis (MoBiTec). pHT43 vector uses the strong promoter preceding the groESL operon of B. subtilis fused to the lac operator allowing induction by addition of IPTG.

The vector encoded a signal peptide from the amyQ gene in frame with the recombinant endoglucanase. Transformation and cultivation of Bacillus transformants was done according to MoBiTec's manual.

Cells were grown overnight in 2xYT medium supplemented with 5 μg/ml chloramphenicol and then transferred into fresh 2xYT medium with 5 μg/ml chloramphenicol at an optical density OD600 of about 0.15. When the cultures reached an OD600 of 0.7-0.8, one mM IPTG was added to induce the expression of recombinant protein. Samples were collected at different time points for analysis during induction (t=2.5 h-48 h).

For the analyses, the culture supernatant was collected and intracellular protein was prepared. For this, the cells were harvested by centrifugation (10 min, 6,000×g, 4° C.), and supernatant was collected for analysis. The cell pellets were washed and resuspend in 50 mM sodium phosphate buffer (pH 7.0) with lysozyme (250 μg/μl). The cells were disrupted by vortexing with glass beads, i.e. bead beating.

Production of the proteins was analyzed using SDS-PAGE, as well as by endoglucanase activity measurements. Active, soluble protein was found only in the supernatant.

Total amount of recombinant protein in the cells and in the supernatant was determined by western blot with anti-His tag antibodies.

For expression in Pichia pastoris, recombinant genes were cloned into a commercial Pichia pastoris expression vector pPICZ-A available from Invitrogen (Life Technologies). This vector provides secreted protein expression under the control of methanol inducible AOX1 promoter upon integration of the construct into genomic DNA of the yeast cell.

Linearized plasmid DNA was introduced into yeast cells by electroporation, and clones with integrated recombinant gene were selected on agar medium plates with Zeocin (25 ug/ml). Ten colonies from each construct were tested in small liquid cultures (3 ml) with 72 hour cultivation in humidified shaker at 28 C according to the Plasmid manufacturer manual (http://tools.lifetechnologies.com/content/sfs/manuals/ppiczalpha_man.pdf). Activity in the medium was measured by azoglucan assay as detailed in the Examples section, and the two best producing clones were selected for each gene. Parallel cultures of the selected clones were gown in flask scale according to the Plasmid manufacturer manual (see above) at 28 degrees C. for 105 hours. Cells were removed by centrifugation, medium containing the recombinant protein was collected. These preparations were used for comparison of specific activities of variant and non-mutated genes.

For recombinant expression in E. coli, recombinant genes were cloned into NcoI-BamH1 sites of pET-22b+ commercial expression vector under the control of T7 bacteriophage promoter, so that recombinant protein is expressed with N-terminal signal peptide PeIB, which is cleaved off from the recombinant polypeptide upon translocation to the periplasmic space. Protein production was carried out in E. coli BL21(DE3) strain according to the plasmid manufacturer protocol http://richsingisercom/4402/Novagen%20pET%20system%20manual.pdf. The incubation temperature for protein production was 30 degrees C., which was found optimal for maximum yield of the active protein. Cells were lysed using lysis buffer (50 mM Tris-HCl pH7.4, 1% Triton X100) and heated at 70 degrees C. for 20 min. Coagulated cell debris was removed by centrifugation. The recombinant endoglucanase being a thermostable protein remained in soluble fraction. Enzymatic activity was detectable only in soluble fraction.

### Example 2

### Construction of Endoglucanase with Improved Properties

Mutations as described herein were introduced into various recombinant genes by standard site-directed mutagenesis essentially as described in WO 2013/038062. In more detail: To introduce mutation P579G into the gene encoding SEQ ID NO: 1, we carried out two separate PCRs:

(1) with Primer1:
(SEQ ID NO: 11)
GAAATTAATACGACTCACTATAGG
and
Primer2-P579:
(SEQ ID NO: 13)
ATATTCGGTCAGACTAATACGATAAC,
(2) with Primer3-P579G:
(SEQ ID NO: 14)
CGTATTAGTCTGACCGAATATggtTGGGGTAGCAATATG
and
Primer4
(SEQ ID NO: 12)
ggttatgctagttattgctcagcggtg.

In both reactions, a recombinant gene encoding SEQ ID NO: 1 was used as the template. Primers 1 and 4 bind inside the vector sequence and are not specific to the recombinant gene. They are therefore used in the mutagenesis procedure of all mutant genes. Primers 2 and 3 are specific for each mutation and bind inside the recombinant gene and their binding sites overlap. Primer 2 binding site is adjacent to the mutation position. The same primer 2 can be used for introducing different mutations at the same position. The binding site of primer 3 contains the mutation site. Primer 3 comprises the mutated (desired) sequence, which is not 100% matching the template (lower case type font in the primer sequence shown in table 6 indicate the mis-matched nucleotides), however, the primer has enough affinity and specificity to the binding site to produce the desired PCR product. Purified PCR products from reactions (1) and (2) were combined and used as template for PCR reaction with Primer 1 and Primer 4. The product of this reaction, containing the mutant sequence of the gene, was cloned in a plasmid vector for expression in E. coli.

For introducing the other mutations, general primers 1 and 4 were used, in combination with specific primers 2 and 3 as listed below in table 4. Mutations of DNA encoding protein with SEQ ID NO: 2 were introduced by the same protocol with corresponding primers, see table 4.

TABLE 4
Sequence of primers used in the mutagenesis procedure.

| SEQ ID NO: | Name | Sequence |
|---|---|---|
| 11 | Primer 1 | GAAATTAATACGACTCACTATAGG |
| 12 | Primer 4 | GGTTATGCTAGTTATTGCTCAGCGGTG |

Primers used in the mutagenesis procedure for SEQ ID NO: 9

| | | |
|---|---|---|
| 13 | Primer2-P579 | ATATTCGGTCAGACTAATACGATAAC |
| 14 | Primer3-P579G | CGTATTAGTCTGACCGAATATggtTGGGGTAGCAATATG |
| 15 | Primer3-P579T | CGTATTAGTCTGACCGAATATaCcTGGGGTAGCAATATG |
| 16 | Primer3-P579S | CGTATTAGTCTGACCGAATATtCcTGGGGTAGCAATATG |
| 17 | Primer3-P579A | CGTATTAGTCTGACCGAATATgCtTGGGGTAGCAATATG |
| 18 | Primer3-P579L | CGTATTAGTCTGACCGAATATCtGTGGGGTAGCAATATG |
| 19 | Primer3-P579I | CGTATTAGTCTGACCGAATATatcTGGGGTAGCAATATG |
| 20 | Primer3-P579V | CGTATTAGTCTGACCGAATATgttTGGGGTAGCAATATG |
| 21 | Primer2-G656 | AACCACCATACCCGGAATGGTT |
| 22 | Primer3-G656V | ACCATTCCGGGTATGGTGGTTGtTGGTCCGAATAG |
| 23 | Primer3-G656S | ACCATTCCGGGTATGGTGGTTtccGGTCCGAATAG |
| 24 | Primer3-G656A | ACCATTCCGGGTATGGTGGTTGcTGGTCCGAATAG |

Primers used in the mutagenesis procedure for SEQ ID NO: 10

| | | |
|---|---|---|
| 25 | Primer2-P579 | CCATTATTTGCAACGGTCATATTGCTACC |
| 26 | Primer3-P579G | CGTACCAGCCTGACCGAATATggtTGGGGTAGCAATATG |
| 27 | Primer3-P579T | CGTACCAGCCTGACCGAATATaCcTGGGGTAGCAATATG |
| 28 | Primer3-P579S | CGTACCAGCCTGACCGAATATtCcTGGGGTAGCAATATG |
| 29 | Primer3-P579A | CGTACCAGCCTGACCGAATATgCtTGGGGTAGCAATATG |
| 30 | Primer3-P579L | CGTACCAGCCTGACCGAATATCtGTGGGGTAGCAATATG |
| 31 | Primer3-P579I | CGTACCAGCCTGACCGAATATatcTGGGGTAGCAATATG |
| 32 | Primer3-P579V | CGTACCAGCCTGACCGAATATgttTGGGGTAGCAATATG |
| 33 | Primer2-G656 | AACAGGATCTTGCAGACCGCTATTCGGACC |
| 34 | Primer3-G656V | ACCATTCCTGGTATGGTTGTTGtTGGTCCGAATAG |
| 35 | Primer3-G656S | ACCATTCCTGGTATGGTTGTTtccGGTCCGAATAG |
| 36 | Primer3-G656A | ACCATTCCTGGTATGGTTGTTGcTGGTCCGAATAG |

### Example 3

### Measurement of Yield

The relative yields of mutated and non-mutated soluble endoglucanases were determined by densitometry of protein bands after denaturing polyacrylamide gel electrophoresis (SDS-PAGE). To this end, samples of soluble proteins after thermal treatment (See example 1) obtained from parallel cultures of mutated and non-mutated clones, were analyzed by gel-electrophoresis under denaturing conditions (a standard method well known in the art of molecular biology). After staining the gel with Coomassie Brilliant Blue, the gel was scanned to obtain a bitmap image, and intensity of the band corresponding to recombinant endoglucanase was quantified by ImageJ software (a public freeware developed at National Institute of Health and online available at http://imagej.nih.gov/ij/). Mutated variants gave virtually identical yield as compared to the corresponding wild type proteins.

### Example 4

### Measuring Relative Endoglucanase Activity in Solution by Azoglucan Assay

The term “endoglucanase activity” is used herein to mean the capability to act as a endoglucanase enzyme, which may be expressed as the maximal initial rate of the specific beta-glucan hydrolysis reaction. Beta glucanase activity was measured using “Assay of endo-β-glucanases using beta-glucazyme tablets” from Megazyme (essentially according to https://secure.megazyme.com/files/Booklet/T-BGZ_DATA.pdf).

Substrate employed was Azurine-crosslinked barley β-glucan (AZCL-Beta-Glucan). The substrate is prepared by dyeing and cross linking highly purified β-glucan to produce a material which hydrates in water but is water insoluble. Hydrolysis by cellulases (endo-1,4-β-glucanase), malt β-glucanase or bacterial 1,3:1,4-β-glucanase (lichenase), produces water soluble dyed fragments, and the rate of release of these (increase in absorbance at 590 nm) can be related directly to enzyme activity. The substrate is supplied commercially, in a ready-to-use tablet form as Beta-Glucazyme Tablets.

All enzymes were prepared in parallel production cultures and processed in the same way, so that volumetric activities could be directly compared.

Enzyme activity assay was performed as follows: Aliquots (0.5 mL) of suitably diluted and buffered to pH 6.0 enzyme were equilibrated in test tubes to 40° C. The reaction was initiated by adding a Beta-Glucazyme Tablet (60 mg). The tablet hydrated rapidly. Exactly 10.0 min after addition of the tablet, 10.0 mL of Trizma Base solution (2% w/v, Sigma Cat. No. T-1503) was added to terminate the reaction. The tube contents were stirred vigorously on a vortex mixer, allowed to stand at room temperature for approx. 5 min, and once again stirred. Then the tubes were centrifuged for 5 min 4 000 g. 300 ul aliquots of the supernatant were transferred to 96-well spectraplates and the absorbance at 590 nm was measured against a substrate blank. Substrate blank was prepared by adding a Beta-Glucazyme tablet to 0.5 mL of the same buffer as in the samples, incubated at 30° Celsius for 10 min, followed by addition of 10.0 mL of Trizma Base (2% w/v) and centrifugation after 5 min.

In order to determine relative activity of mutated endoglucanases, the absorbance of the reference endoglucanase sample was taken as 100%, and relative activity was determined as fraction of this absorbance. As recombinant enzyme yield was virtually identical in the mutant proteins as compared to the respective wild-type according to SEQ ID NO: 1 or SEQ ID NO: 2 (see example 3), the relative activities measured in this example represent specific relative activities, in other words, the specific activity of the mutants was higher than the specific activity of the wild-type enzyme.

### REFERENCES

1. Methods in Molecular Biology, Vol 182, “In vitro mutagenesis protocols”, Eds Jeff Braman, Humana Press 2002).

1. A polypeptide with endoglucanase activity (EC 3.2.1.4) comprising an amino acid sequence that is at least 90% identical to the amino acid sequence according to SEQ ID NO: 1 or SEQ ID NO: 2, wherein the polypeptide comprises an amino acid selected from the group consisting of Glycine, Threonine, Serine, Alanine, Leucine, Isoleucine and Valine at a position corresponding to position 579 in SEQ ID NO: 1 or SEQ ID NO: 2 or an amino acid selected from the group consisting of Serine, Alanine and Valine at a position corresponding to position 656 in SEQ ID NO: 1 or SEQ ID NO: 2.

2. The polypeptide of claim 1, wherein the polypeptide comprises an amino acid selected from the group consisting of Glycine, Threonine, Serine, Alanine, Leucine, Isoleucine and Valine at a position corresponding to position 579 in SEQ ID NO: 1 or SEQ ID NO: 2 and an amino acid selected from the group consisting of Serine, Alanine and Valine at a position corresponding to position 656 in SEQ ID NO: 1 or SEQ ID NO: 2.

3. The polypeptide of claim 1 which is produced in E. coli.

4. The polypeptide of claim 1 wherein the polypeptide is an isolated polypeptide.

5. The polypeptide of claim 1, wherein the polypeptide is comprised in a composition.

6. A nucleic acid encoding the polypeptide according to claim 1.

7. The nucleic acid according to of claim 6, wherein the nucleic acid is in a vector.

8. The nucleic acid of claim 6, wherein the nucleic acid is comprised in a composition.

9. The nucleic acid of claim 6, wherein the nucleic acid is comprised in a recombinant host cell.

10. The nucleic acid of claim 9, wherein the host cell is selected from the group consisting of Escherichia coli, Bacillus, Corynebacterium, Pseudomonas, Pichia pastoris, Saccharomyces cerevisiae, Yarrowia lipolytica, filamentous fungi, yeast and insect cells.

11. (canceled)

12. (canceled)

13. Method for improving the specific activity of a polypeptide with endoglucanase activity in a heterologous expression system comprising a step of altering an amino acid at a position corresponding to position 579 into a Glycine, Threonine, Serine, Alanine, Leucine, Isoleucine or Valine and/or altering an amino acid at a position corresponding to position 656 into a Serine, Alanine or Valine, wherein the polypeptide with endoglucanase activity is a polypeptide with an amino acid sequence according to SEQ ID NO: 1 or SEQ ID NO: 2 or a polypeptide with an amino acid sequence that is at least 90% identical to the sequence of SEQ ID NO: 1 or SEQ ID NO: 2.


SEQUENCE LISTING (36 sequences)

SEQ ID NO: 1 - protein, 709 aa, Thermotoga
MVPEGGGKAPPEEAGETAAEELLENGDFSAGTTPWTLFTQDPGKAVMTTEDGMLVFEISSIGEDTWHVQPGYPGLHLEEGHTYRLEFDMRASEPRTVQVRIQKDGPPWTGYLEENFQVGTALQHYTFEFSMQETDKAARLVFNLGTAAEGTAPAGAHRIYLDNISLKDLTGGPPEEKTGGLRPAVHLNQVGYLPTAPKVFVTMVDSSSFKVLEADSGKEVFSGTLSSPIPDRDSGDTVRLGDFTALTTPGTYVVEVGETRSVPFEIREDIYEELHTALFRFFYLQRCGTELAPSLAGAWAHPACHTTPALVYGTTITKEVRGGWHDAGDYGRYVVPAAKAVADLLLAHLFYPNATSSDALEIPESGNGVPDVLDEVKDELLWLLSMQDPESGGVYHKVTTRNFAGFDWPHITGGELVLSPISSAATADFAAVMAMASRVYAIVEPRLARRALEAARRAWSWLEAHPSAPGFRNPPGIQTGEYGDDHDADERYWAACELYAATGEEDFHTALKALSRGDIRAGLGWADVADYGTITYLFFTPHKDEALAAELAAHLTAKAEAILATMETSGYRISLTEYPWGSNMTVANNGMYLLIASRLTGDPRYERAAAEHLDYLLGRNPLSRSYITGFGEKAAEHPHHRVSSAAGTTIPGMVVGGPNSGLQDPVAQGALRGEPPAKCYIDDVGSYSTNEVTIYWNSPVYFLVSGLVE

SEQ ID NO: 2 - protein, 709 aa, Thermotoga
MVPERRGEAPPEEAGETAAEELLENGDFSAGTTPWTLFTQDPGKAVMTTEDGMLVFEISSIGEDTWHVQPGYPGLHLIEGHTYRLEFDMRASEPRTVQVRIQKDGPPWTGYLEENFQVGNEFQHYTFEFTMQETDKAARLVFNLGTAAEGTAPAGPHRVSLDNISLKDLTGGPPEEETGGLRPAVHLNQLGYLPTAPKVFVTTVDSSSFKVLEADSGKEVFSGTLSSPIPDRDSGDTVRVGDFTALTTPGTYVVEAGETRSVPFEIREDIYEELHTALFRFFYLQRCGTELASSLAGAWAHPACHTAPALVYGTTITKEVRGGWHDAGDYGRYVVPAAKAVADIILAHLFYPDATSSDALEIPESGNGVPDVLDEVRDELLWLLSMQDPEGGGVYHKVTTRNFAGFDWPQNTGGELVLSPISPTATADFAAVMAMASRVYASIDPGLARRALEAARRAWSWLEAHPSAPGFRNPPGIQTGEYGDDHDADERYWAACELYAATGEEDFHAALEGLSRGDLRAGLGWADVADYGTITYLFFTPHKDEALAAELAAHLTDKAEAILATMETSGYRTSLTEYPWGSNMTVANNGMYLLITSRLTGDPRYERAAAEHLDYLLGRNPLSRSYITGFGEKVAEHPHHRVSSAAGMTIPGMVVGGPNSGLQDPVAQGALRGEPPAKCYIDDVGSYSTNEVTIYWNSPVYFLVSGLVE

SEQ ID NO: 3 - protein, 709 aa, Thermotoga
  MISC_FEATURE (579)..(579): Xaa is Glycine, Threonine, Serine, Alanine, Leucine, Isoleucine or Valine
  MISC_FEATURE (656)..(656): Xaa is Serine, Valine or Alanine.
MVPEGGGKAPPEEAGETAAEELLENGDFSAGTTPWTLFTQDPGKAVMTTEDGMLVFEISSIGEDTWHVQPGYPGLHLEEGHTYRLEFDMRASEPRTVQVRIQKDGPPWTGYLEENFQVGTALQHYTFEFSMQETDKAARLVFNLGTAAEGTAPAGAHRIYLDNISLKDLTGGPPEEKTGGLRPAVHLNQVGYLPTAPKVFVTMVDSSSFKVLEADSGKEVFSGTLSSPIPDRDSGDTVRLGDFTALTTPGTYVVEVGETRSVPFEIREDIYEELHTALFRFFYLQRCGTELAPSLAGAWAHPACHTTPALVYGTTITKEVRGGWHDAGDYGRYVVPAAKAVADLLLAHLFYPNATSSDALEIPESGNGVPDVLDEVKDELLWLLSMQDPESGGVYHKVTTRNFAGFDWPHITGGELVLSPISSAATADFAAVMAMASRVYAIVEPRLARRALEAARRAWSWLEAHPSAPGFRNPPGIQTGEYGDDHDADERYWAACELYAATGEEDFHTALKALSRGDIRAGLGWADVADYGTITYLFFTPHKDEALAAELAAHLTAKAEAILATMETSGYRISLTEYXWGSNMTVANNGMYLLIASRLTGDPRYERAAAEHLDYLLGRNPLSRSYITGFGEKAAEHPHHRVSSAAGTTIPGMVVXGPNSGLQDPVAQGALRGEPPAKCYIDDVGSYSTNEVTIYWNSPVYFLVSGLVE

SEQ ID NO: 4 - protein, 709 aa, Thermotoga
  MISC_FEATURE (579)..(579): Xaa is Glycine, Threonine, Serine, Alanine, Leucine, Isoleucine or Valine
  MISC_FEATURE (656)..(656): Xaa is Serine, Valine or Alanine
MVPERRGEAPPEEAGETAAEELLENGDFSAGTTPWTLFTQDPGKAVMTTEDGMLVFEISSIGEDTWHVQPGYPGLHLIEGHTYRLEFDMRASEPRTVQVRIQKDGPPWTGYLEENFQVGNEFQHYTFEFTMQETDKAARLVFNLGTAAEGTAPAGPHRVSLDNISLKDLTGGPPEEETGGLRPAVHLNQLGYLPTAPKVFVTTVDSSSFKVLEADSGKEVFSGTLSSPIPDRDSGDTVRVGDFTALTTPGTYVVEAGETRSVPFEIREDIYEELHTALFRFFYLQRCGTELASSLAGAWAHPACHTAPALVYGTTITKEVRGGWHDAGDYGRYVVPAAKAVADIILAHLFYPDATSSDALEIPESGNGVPDVLDEVRDELLWLLSMQDPEGGGVYHKVTTRNFAGFDWPQNTGGELVLSPISPTATADFAAVMAMASRVYASIDPGLARRALEAARRAWSWLEAHPSAPGFRNPPGIQTGEYGDDHDADERYWAACELYAATGEEDFHAALEGLSRGDLRAGLGWADVADYGTITYLFFTPHKDEALAAELAAHLTDKAEAILATMETSGYRTSLTEYXWGSNMTVANNGMYLLITSRLTGDPRYERAAAEHLDYLLGRNPLSRSYITGFGEKVAEHPHHRVSSAAGMTIPGMVVXGPNSGLQDPVAQGALRGEPPAKCYIDDVGSYSTNEVTIYWNSPVYFLVSGLVE

SEQ ID NO: 5 - protein, 709 aa, Thermotoga
  MISC_FEATURE (579)..(579): Xaa is Glycine, Threonine, Serine, Alanine, Leucine, Isoleucine or Valine
MVPEGGGKAPPEEAGETAAEELLENGDFSAGTTPWTLFTQDPGKAVMTTEDGMLVFEISSIGEDTWHVQPGYPGLHLEEGHTYRLEFDMRASEPRTVQVRIQKDGPPWTGYLEENFQVGTALQHYTFEFSMQETDKAARLVFNLGTAAEGTAPAGAHRIYLDNISLKDLTGGPPEEKTGGLRPAVHLNQVGYLPTAPKVFVTMVDSSSFKVLEADSGKEVFSGTLSSPIPDRDSGDTVRLGDFTALTTPGTYVVEVGETRSVPFEIREDIYEELHTALFRFFYLQRCGTELAPSLAGAWAHPACHTTPALVYGTTITKEVRGGWHDAGDYGRYVVPAAKAVADLLLAHLFYPNATSSDALEIPESGNGVPDVLDEVKDELLWLLSMQDPESGGVYHKVTTRNFAGFDWPHITGGELVLSPISSAATADFAAVMAMASRVYAIVEPRLARRALEAARRAWSWLEAHPSAPGFRNPPGIQTGEYGDDHDADERYWAACELYAATGEEDFHTALKALSRGDIRAGLGWADVADYGTITYLFFTPHKDEALAAELAAHLTAKAEAILATMETSGYRISLTEYXWGSNMTVANNGMYLLIASRLTGDPRYERAAAEHLDYLLGRNPLSRSYITGFGEKAAEHPHHRVSSAAGTTIPGMVVGGPNSGLQDPVAQGALRGEPPAKCYIDDVGSYSTNEVTIYWNSPVYFLVSGLVE

SEQ ID NO: 6 - protein, 709 aa, Thermotoga
  MISC_FEATURE (656)..(656): Xaa is Serine, Valine or Alanine
MVPEGGGKAPPEEAGETAAEELLENGDFSAGTTPWTLFTQDPGKAVMTTEDGMLVFEISSIGEDTWHVQPGYPGLHLEEGHTYRLEFDMRASEPRTVQVRIQKDGPPWTGYLEENFQVGTALQHYTFEFSMQETDKAARLVFNLGTAAEGTAPAGAHRIYLDNISLKDLTGGPPEEKTGGLRPAVHLNQVGYLPTAPKVFVTMVDSSSFKVLEADSGKEVFSGTLSSPIPDRDSGDTVRLGDFTALTTPGTYVVEVGETRSVPFEIREDIYEELHTALFRFFYLQRCGTELAPSLAGAWAHPACHTTPALVYGTTITKEVRGGWHDAGDYGRYVVPAAKAVADLLLAHLFYPNATSSDALEIPESGNGVPDVLDEVKDELLWLLSMQDPESGGVYHKVTTRNFAGFDWPHITGGELVLSPISSAATADFAAVMAMASRVYAIVEPRLARRALEAARRAWSWLEAHPSAPGFRNPPGIQTGEYGDDHDADERYWAACELYAATGEEDFHTALKALSRGDIRAGLGWADVADYGTITYLFFTPHKDEALAAELAAHLTAKAEAILATMETSGYRISLTEYPWGSNMTVANNGMYLLIASRLTGDPRYERAAAEHLDYLLGRNPLSRSYITGFGEKAAEHPHHRVSSAAGTTIPGMVVXGPNSGLQDPVAQGALRGEPPAKCYIDDVGSYSTNEVTIYWNSPVYFLVSGLVE

SEQ ID NO: 7 - protein, 709 aa, Thermotoga
  MISC_FEATURE (579)..(579): Xaa is Glycine, Threonine, Serine, Alanine, Leucine, Isoleucine or Valine
MVPERRGEAPPEEAGETAAEELLENGDFSAGTTPWTLFTQDPGKAVMTTEDGMLVFEISSIGEDTWHVQPGYPGLHLIEGHTYRLEFDMRASEPRTVQVRIQKDGPPWTGYLEENFQVGNEFQHYTFEFTMQETDKAARLVFNLGTAAEGTAPAGPHRVSLDNISLKDLTGGPPEEETGGLRPAVHLNQLGYLPTAPKVFVTTVDSSSFKVLEADSGKEVFSGTLSSPIPDRDSGDTVRVGDFTALTTPGTYVVEAGETRSVPFEIREDIYEELHTALFRFFYLQRCGTELASSLAGAWAHPACHTAPALVYGTTITKEVRGGWHDAGDYGRYVVPAAKAVADIILAHLFYPDATSSDALEIPESGNGVPDVLDEVRDELLWLLSMQDPEGGGVYHKVTTRNFAGFDWPQNTGGELVLSPISPTATADFAAVMAMASRVYASIDPGLARRALEAARRAWSWLEAHPSAPGFRNPPGIQTGEYGDDHDADERYWAACELYAATGEEDFHAALEGLSRGDLRAGLGWADVADYGTITYLFFTPHKDEALAAELAAHLTDKAEAILATMETSGYRTSLTEYXWGSNMTVANNGMYLLITSRLTGDPRYERAAAEHLDYLLGRNPLSRSYITGFGEKVAEHPHHRVSSAAGMTIPGMVVGGPNSGLQDPVAQGALRGEPPAKCYIDDVGSYSTNEVTIYWNSPVYFLVSGLVE

SEQ ID NO: 8 - protein, 709 aa, Thermotoga
  MISC_FEATURE (656)..(656): Xaa is Serine, Valine or Alanine
MVPERRGEAPPEEAGETAAEELLENGDFSAGTTPWTLFTQDPGKAVMTTEDGMLVFEISSIGEDTWHVQPGYPGLHLIEGHTYRLEFDMRASEPRTVQVRIQKDGPPWTGYLEENFQVGNEFQHYTFEFTMQETDKAARLVFNLGTAAEGTAPAGPHRVSLDNISLKDLTGGPPEEETGGLRPAVHLNQLGYLPTAPKVFVTTVDSSSFKVLEADSGKEVFSGTLSSPIPDRDSGDTVRVGDFTALTTPGTYVVEAGETRSVPFEIREDIYEELHTALFRFFYLQRCGTELASSLAGAWAHPACHTAPALVYGTTITKEVRGGWHDAGDYGRYVVPAAKAVADIILAHLFYPDATSSDALEIPESGNGVPDVLDEVRDELLWLLSMQDPEGGGVYHKVTTRNFAGFDWPQNTGGELVLSPISPTATADFAAVMAMASRVYASIDPGLARRALEAARRAWSWLEAHPSAPGFRNPPGIQTGEYGDDHDADERYWAACELYAATGEEDFHAALEGLSRGDLRAGLGWADVADYGTITYLFFTPHKDEALAAELAAHLTDKAEAILATMETSGYRTSLTEYPWGSNMTVANNGMYLLITSRLTGDPRYERAAAEHLDYLLGRNPLSRSYITGFGEKVAEHPHHRVSSAAGMTIPGMVVXGPNSGLQDPVAQGALRGEPPAKCYIDDVGSYSTNEVTIYWNSPVYFLVSGLVE

SEQ ID NO: 9 - DNA, 2127 bp, Thermotoga
atggttccggaaggtggtggtaaagcaccgcctgaagaagcaggcgaaaccgcagccgaagaactgctggaaaatggtgattttagcgcaggcaccaccccgtggaccctgtttacccaagatccgggtaaagcagttatgaccaccgaagatggtatgctggtttttgaaattagcagcattggtgaagatacctggcatgttcagcctggttatccgggtctgcatctggaagaaggtcatacctatcgtctggaatttgatatgcgtgcaagcgaaccgcgtaccgttcaggttcgtattcagaaagatggtccgccttggaccggttacctggaagaaaactttcaggttggcaccgcactgcagcattatacctttgaatttagcatgcaagaaaccgataaagcagcacgtctggtttttaatctgggcaccgcagcagaaggtacagcaccggcaggcgcacatcgtatttatctggataacattagcctgaaagatctgaccggtggtcctccggaagaaaaaaccggtggcctgcgtccggcagttcatctgaatcaggttggttatctgccgaccgcaccgaaagtttttgttacaatggttgatagcagcagcttcaaagttctggaagcagatagcggtaaagaagtttttagcggcaccctgagcagcccgattccggatcgtgatagcggtgataccgttcgtctgggcgattttaccgcactgaccacaccgggtacatatgttgttgaagttggtgaaacccgtagcgttccgtttgaaattcgtgaagatatctatgaggaactgcatacagcactgttccgttttttctatctgcagcgttgtggcaccgaactggcaccgagcctggctggtgcatgggcacatccggcatgtcatacaacaccggcactggtttatggcaccaccattaccaaagaagttcgcggaggttggcatgatgccggtgattatggtcgttatgttgttccggcagcaaaagcagttgccgatctgctgctggcacacctgttttatccgaatgcaaccagcagtgatgcactggaaattccggaaagcggtaatggcgttccggatgttctggatgaagttaaagatgagctgctgtggctgctgagcatgcaggaccctgaaagcggtggtgtttatcataaagttaccacccgtaattttgccggttttgattggcctcatattaccggtggtgaactggttctgagcccgattagcagcgcagcaaccgcagattttgcagccgttatggcaatggcaagccgtgtttatgcaattgttgaaccgcgtctggcacgtcgtgccctggaagctgctcgtcgtgcatggtcatggctggaagcacatccgagcgcaccgggttttcgtaatccgcctggtattcagacaggtgaatatggtgatgatcatgatgcggatgaacgttattgggcagcatgtgaactgtatgccgcaaccggtgaagaagattttcatacagcgctgaaagcactgagccgtggtgatattcgtgcaggtctgggttgggcagatgttgcagattatggtacaattacctacctgtttttcacaccgcataaagatgaagcactggcagcagaactggcagcccatctgaccgcaaaagccgaagcaattctggcaacaatggaaaccagcggttatcgtattagtctgaccgaatatccgtggggtagcaatatgaccgttgcaaataatggtatgtatctgctgattgcaagccgtctgacaggtgatccgcgttatgaacgtgcagcagccgaacatctggattatctgctgggtcgcaatccgctgagccgtagctatattacaggttttggtgaaaaagctgcggaacatcctcatcatcgtgtttcaagcgcagccggtacaaccattccgggtatggtggttggtggtccgaatagcggtctgcaagatcctgttgcacagggtgcactgcgtggtgaaccgcctgcaaaatgttatattgatgatgttggtagctacagcaccaacgaagttaccatttattggaatagtccggtgtattttctggtgagcggtctggttgaa

SEQ ID NO: 10 - DNA, 2127 bp, Thermotoga
atggttccggaacgtcgtggtgaagcaccgcctgaagaagcaggcgaaaccgcagccgaagaactgctggaaaatggtgattttagcgcaggtacaaccccgtggacactgtttacccaagatccgggtaaagcagttatgaccaccgaagatggtatgctggtttttgaaattagcagcattggtgaagatacctggcatgttcagcctggttatccgggtctgcatctgattgaaggtcatacctatcgtctggaatttgatatgcgtgcaagcgaaccgcgtaccgttcaggttcgtattcagaaagatggtccgccttggaccggttatctggaagaaaactttcaggtgggtaacgaatttcagcactatacctttgaatttaccatgcaagaaaccgataaagcagcacgtctggtttttaatctgggcaccgcagcagaaggcaccgcaccggcaggtccgcatcgtgttagcctggataacattagcctgaaagatctgaccggtggtcctccggaagaagaaacaggcggtctgcgtccggcagttcatctgaatcagctgggttatctgccgaccgcaccgaaagtgtttgttaccaccgttgatagcagcagctttaaagttctggaagcagatagcggtaaagaagtttttagcggcaccctgagcagcccgattccggatcgtgatagcggtgataccgttcgtgttggcgattttaccgcactgaccacaccgggtacatatgttgttgaagccggtgaaacccgtagcgttccgtttgaaattcgcgaagatatttatgaggaactgcatacagcactgttccgctttttctatctgcagcgttgtggcaccgaactggcaagcagcctggcaggcgcatgggcacatccggcatgtcatacggcaccggcactggtttatggcaccaccattaccaaagaagttcgcggaggttggcatgatgccggtgattatggtcgttatgttgttccggcagcaaaagcagttgcagatattattctggcacacctgttttatccggatgcaaccagcagtgatgcactggaaattccggaaagcggtaatggcgttccggatgttctggatgaagttcgtgatgagctgctgtggctgctgagcatgcaggaccctgaaggtggtggtgtttatcataaagttaccacacgtaactttgccggttttgattggcctcagaataccggtggtgaactggttctgagcccgattagcccgaccgcaaccgcagattttgcagccgttatggcaatggcaagccgtgtttatgcaagcattgatcctggtctggcacgtcgtgccctggaagctgctcgtcgtgcatggtcatggctggaagcacatccgagcgcaccgggttttcgtaatccgcctggtattcagacaggtgaatatggtgatgatcatgatgcggatgaacgttattgggcagcatgtgaactgtatgccgcaaccggtgaagaagattttcacgcagcactggaaggtctgagccgtggtgatctgcgtgcaggtttaggttgggcagatgttgcagattatggtacaattacctacctgtttttcacaccgcataaagatgaagcactggcagcagaactggcagcccatctgacagataaagccgaagcaattctggcaacaatggaaaccagcggttatcgtaccagcctgaccgaatatccgtggggtagcaatatgaccgttgcaaataatggtatgtatctgctgattaccagccgtctgacaggtgatccgcgttatgaacgtgcagcagccgaacatctggattatctgctgggtcgcaatccgctgagccgtagctatattaccggttttggtgaaaaagttgcggaacatcctcatcatcgtgtgagcagcgcagccggtatgaccattcctggtatggttgttggtggtccgaatagcggtctgcaagatcctgttgcacagggtgcactgcgtggtgaaccgcctgcaaaatgttatattgatgatgttggtagctacagcaccaacgaagttaccatttattggaatagtccggtgtattttctggtgagcggtctggttgaa

SEQ ID NO: 11 - DNA, 24 bp, Thermotoga
gaaattaatacgactcactatagg

SEQ ID NO: 12 - DNA, 27 bp, Thermotoga
ggttatgctagttattgctcagcggtg

SEQ ID NO: 13 - DNA, 26 bp, Thermotoga
atattcggtcagactaatacgataac

SEQ ID NO: 14 - DNA, 39 bp, Thermotoga
cgtattagtctgaccgaatatggttggggtagcaatatg

SEQ ID NO: 15 - DNA, 39 bp, Thermotoga
cgtattagtctgaccgaatatacctggggtagcaatatg

SEQ ID NO: 16 - DNA, 39 bp, Thermotoga
cgtattagtctgaccgaatattcctggggtagcaatatg

SEQ ID NO: 17 - DNA, 39 bp, Thermotoga
cgtattagtctgaccgaatatgcttggggtagcaatatg

SEQ ID NO: 18 - DNA, 39 bp, Thermotoga
cgtattagtctgaccgaatatctgtggggtagcaatatg

SEQ ID NO: 19 - DNA, 39 bp, Thermotoga
cgtattagtctgaccgaatatatctggggtagcaatatg

SEQ ID NO: 20 - DNA, 39 bp, Thermotoga
cgtattagtctgaccgaatatgtttggggtagcaatatg

SEQ ID NO: 21 - DNA, 22 bp, Thermotoga
aaccaccatacccggaatggtt

SEQ ID NO: 22 - DNA, 35 bp, Thermotoga
accattccgggtatggtggttgttggtccgaatag

SEQ ID NO: 23 - DNA, 35 bp, Thermotoga
accattccgggtatggtggtttccggtccgaatag

SEQ ID NO: 24 - DNA, 35 bp, Thermotoga
accattccgggtatggtggttgctggtccgaatag

SEQ ID NO: 25 - DNA, 29 bp, Thermotoga
ccattatttgcaacggtcatattgctacc

SEQ ID NO: 26 - DNA, 39 bp, Thermotoga
cgtaccagcctgaccgaatatggttggggtagcaatatg

SEQ ID NO: 27 - DNA, 39 bp, Thermotoga
cgtaccagcctgaccgaatatacctggggtagcaatatg

SEQ ID NO: 28 - DNA, 39 bp, Thermotoga
cgtaccagcctgaccgaatattcctggggtagcaatatg

SEQ ID NO: 29 - DNA, 39 bp, Thermotoga
cgtaccagcctgaccgaatatgcttggggtagcaatatg

SEQ ID NO: 30 - DNA, 39 bp, Thermotoga
cgtaccagcctgaccgaatatctgtggggtagcaatatg

SEQ ID NO: 31 - DNA, 39 bp, Thermotoga
cgtaccagcctgaccgaatatatctggggtagcaatatg

SEQ ID NO: 32 - DNA, 39 bp, Thermotoga
cgtaccagcctgaccgaatatgtttggggtagcaatatg

SEQ ID NO: 33 - DNA, 30 bp, Thermotoga
aacaggatcttgcagaccgctattcggacc

SEQ ID NO: 34 - DNA, 35 bp, Thermotoga
accattcctggtatggttgttgttggtccgaatag

SEQ ID NO: 35 - DNA, 35 bp, Thermotoga
accattcctggtatggttgtttccggtccgaatag

SEQ ID NO: 36 - DNA, 35 bp, Thermotoga
accattcctggtatggttgttgctggtccgaatag